(19) United States
(12) Patent Application Publication
(10) Pub. No.: US 2017/0088814 A1
(43) Pub. Date: Mar. 30, 2017

(54) PHYSICAL SELF-ORGANIZING HYDROGEL SYSTEM FOR BIOTECHNOLOGICAL APPLICATIONS

(71) Applicants: LEIBNIZ-INSTITUTE FÜR POLYMERFORSCHUNG DRESDEN E.V., 01069 Dresden (DE); TECHNISCHE UNIVERSITÄT DRESDEN, 01069 Dresden (DE)

(72) Inventors: Michael Thompson, Dresden (DE); Robert Wieduwild, Dresden (DE); Francesco Reddavide, Dresden (SE); Mikhail Tsurkan, Dresden (DE); Helena Andrade, Dresden (DE); Carsten Wermer, Dresden (DE); Yixin Zhang, Dresden (DE)

(21) Appl. No.: 15/301,038

(22) PCT Filed: Mar. 27, 2015
(86) PCT No.: PCT/DE2015/100130

§ 371 Date: Dec. 19, 2016

(30) Foreign Application Priority Data
Apr. 1, 2014 (DE) 10 2014 104 566.1

(51) Int. Cl. C12N 5/00

(52) U.S. Cl. CPC C12N 5/0068; C12N 2533/20; C12N 2533/30; C12N 2533/50

(57) ABSTRACT

A physical self-organizing hydrogel system for biotechnological applications composed of a non-covalent network on the basis of a protein-ligand interaction includes a tetrameric protein and biotin or a derivative thereof as a ligand, wherein biotin or derivative is covalently conjugated to an end of a polymer chain or a linear or multi-arm synthetic polymer of a single-or double-strand oligonucleotide and wherein the solid content in relation to the entire hydrogel is at least 3% and the conjugates are crosslinked by the tetrameric protein. A mixture ratio is a molar equivalent ratio between the protein and the number of terminal biotinylated ends of the polymer chains in the biotin-polymer conjugate of 1:2 to 1:8. The hydrogel formation occurs with controlled kinetics on the basis of protein-ligand interaction.

The present invention refers to a physical self-organizing hydrogel system for biotechnology applications. The hydrogel formation takes place under controlled kinetics based on a protein-ligand-interaction.

The avidin/streptavidin-biotin-interaction as the protein-ligand-pair is most often utilized in the field of biotechnology. Biomaterials based on avidin/streptavidin-biotin-interaction are used, for example, in a cell culture or in controlled release of active ingredients. To date, chemical reactions are utilized in research to incorporate biotin or streptavidin into a material or onto a surface. Recently, a covalent hydrogel-system was developed that is combined with a 2-photon-photochemical reaction. This method permits inclusion of biotinylated substances in 3-D matrices. For many cell-based application methods that forgo chemical reactions are advantageous for integrating biofunctions.

Chemical reactions across a wide scope are difficult to control and yield very little. Even though it is also very difficult to form a physical hydrogel, which for instance is based on an avidin/streptavidin-biotin interaction, as these components in compounds often tend to precipitate and aggregate.

Object of the present invention is to provide conditions that prevent the generation of small polymer particles and to realize the formation of a coherent hydrogel or a biofilm.

Solution of this object of the present invention refers to a physical self-organizing hydrogel system for biotechnological applications according to claim 1 consisting of a non-covalent network on the basis of a protein-ligand-interaction and comprises as ligand, biotin or one of its derivatives, wherein biotin or one of its derivatives each is covalently conjugated at an end of a polymer chain of a linear or multi-arm synthetic polymer or a single-or double strand-oligonucleotide and as protein, a tetrameric protein, whereby the solids content relative to the entire hydrogel is at least 3, preferably at least 4% and the conjugates are cross-linked by the tetrameric protein. According to the present invention, a mixing ratio of 1:2 to 1:8 is contemplated as equivalent ratio between the protein and the number of biotinylated terminal ends of the polymer chain in the biotin-polymer-conjugate. Preferred is an equivalent ratio of 1:4, which at two terminal ends per polymer chain, such as for example at a conjugate of 5 kDa linear polyethylene glycol (PEG) with biotin, corresponds to a molar ratio of 1:2 between protein and polymer-biotin-conjugate. Especially advantageous is a solids content of at least 6%, preferably at least 9%. avidin, streptavidin or a mutant of these proteins is preferred as a tetrameric conjugate linking protein. Thereby, it is preferred that none of the several proteins are covalently linked with each other.

The network formation is controlled by the processes of the physical-chemical interaction between the polymer chains. Surprisingly, the selected molecule design and the cross-linking conditions according to the present invention lead to the prevention of small polymer particles such that the formation of a connected hydrogel or a bio-matrix-film can be realized.

With the advantageous use of traptavidin, a mutated form of streptavidin, the hydrogel-stability can be additionally markedly increased as compared to streptavidin/avidin-polymer-biotin-conjugates systems.

In another embodiment of the present invention the polymer chain is formed from linear or multi-arm polyethylene glycol (PEG). If an oligonucleotide is contemplated as the polymer chain, according to a further advantageous embodiment, a branched oligonucleotide that is, a nucleotide having more than two ends can be utilized. In an especially preferred embodiment of the present invention, the oligonucleotide-polymer chain consists of a double-stranded DNA. In a preferred embodiment of the present invention, instead of the synthetic polymer chain or a single-stranded oligonucleotide, double-stranded DNA can be utilized. With this a more controlled gelation process can be realized.

The optimized methods improve not only the hydrogel stability but also make the formation of thin hydrogel films with minimal material input possible. Thus, the system can be used to form thin hydrogel films at cost-effective production in order to realize the coating of large surfaces with the desired biomaterials. The hydrogel system thus forms a simple platform, to produce many biomatrix films with different conditions and therefore be able to carry out, for example, a high throughput-screening. A further interesting feature of the novel hydrogel system is its capacity to have it reconstituted entirely after drying. This characteristic makes for an easy transport of the material.

The hydrogel according to the present invention can be formed in situ without chemical reactions. This is novel as compared to the conventional hydrogels based on streptavidin-biotin-interactions. The erosion can be accelerated by the addition of biotin without proteases. One possible application is in the analysis of intact extracellular matrix proteins that are segregated form cells embedded in the hydrogel.

A further aspect of the present invention refers to a cross linking method based on a biotin-avidin-interaction. The network formation is being controlled through the processes of the physical-chemical interaction between the polymer chains. As afore-stated, a molecule design and the cross-linking conditions could be determined that leads to the prevention of producing small polymer particles and realizes the formation of a coherent hydrogel or a bio-matrix film. With the method of the present invention for producing the hydrogel, including all described embodiments, the components to be cross-linked, that is, the protein and the conjugate, at a solids content of at least 3% and in an equivalent ratio of 1:2 to 1:8 are mixed together. Mixing of the two components is done preferably in a high speed rotary mixer to prevent formation of particles rather than the hydrogel formation.

The low molecular ligand biotin or one of its derivatives, such as for example, iminobiotin or desthiobiotin is being conjugated at the ends of the polymer chain. The polymer can be a linear synthetic polymer or a single/double strand-oligonucleotide. The polymer can also be a multi-arm synthetic polymer or a branched oligonucleotide structure which has more than two terminal ends. The resulting polymer-biotin-conjugates are crosslinked by a tetrameric protein. The tetrameric protein can be streptavidin or avidin or mutants of streptavidin and avidin. Especially preferred is the streptavidin mutant traptavidin that exhibits a higher affinity to biotin as compared to streptavidin.

The biomaterial systems can be further modified through either insertion of peptide sequences into the polymer chain or by the addition of biotinylated peptides, biotinylated active ingredients, biotinylated oligosaccharides, biotinylated oligonucleotides or biotinylated proteins into the hydrogel. The biotin group, as afore-stated can be replaced by biotin analogs, desthiobiotin or iminobiotin, of which it is expected that they raise the rates of release of peptides, active ingredients, oligosaccharides oligonucleotides and proteins. Furthermore, the afore-stated various covalent and non-covalent modifications can also be combined, thus producing an unlimited number of new biomaterials which can be utilized for screening and optimizing of biomaterials by a combinational approach.

A further aspect of the present invention refers to the use of a physical self-organizing hydrogel system for a cell culture; that is, the resulting biomaterial systems can be utilized for cultivating cells on the biomaterials as well as the encapsulation of cells into the hydrogel.

Further details, features and advantages of the invention emerge from the following description of exemplary embodiments with reference to the associated drawings, in which:

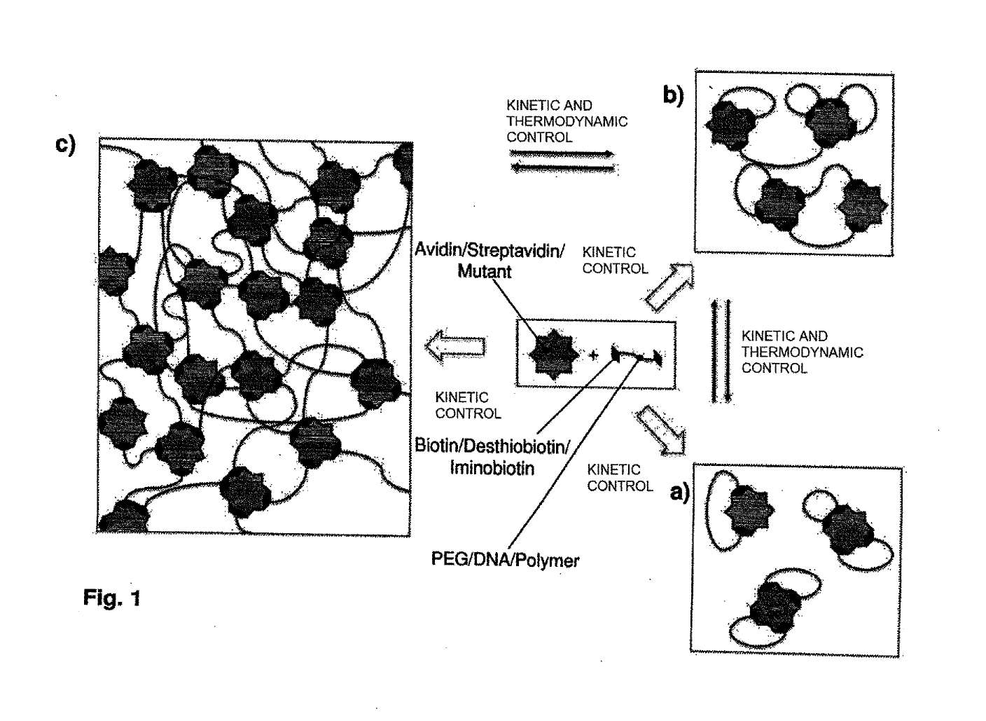

FIG. 1 is a schematic illustration of the kinetic and thermodynamic control of crosslinking formation,

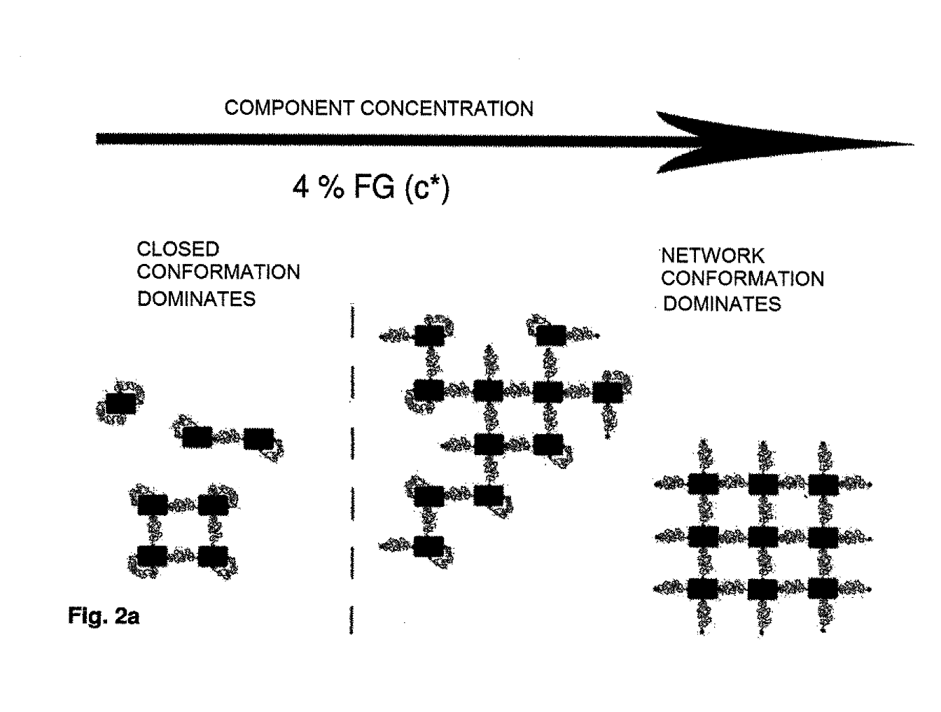

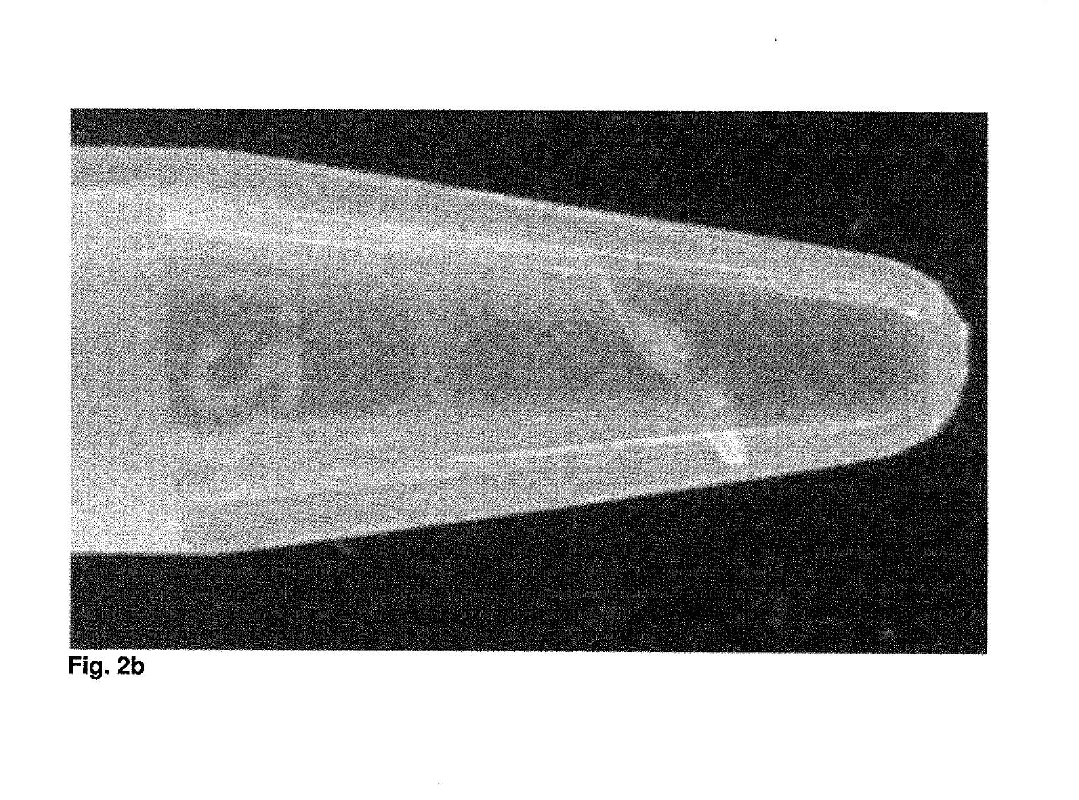

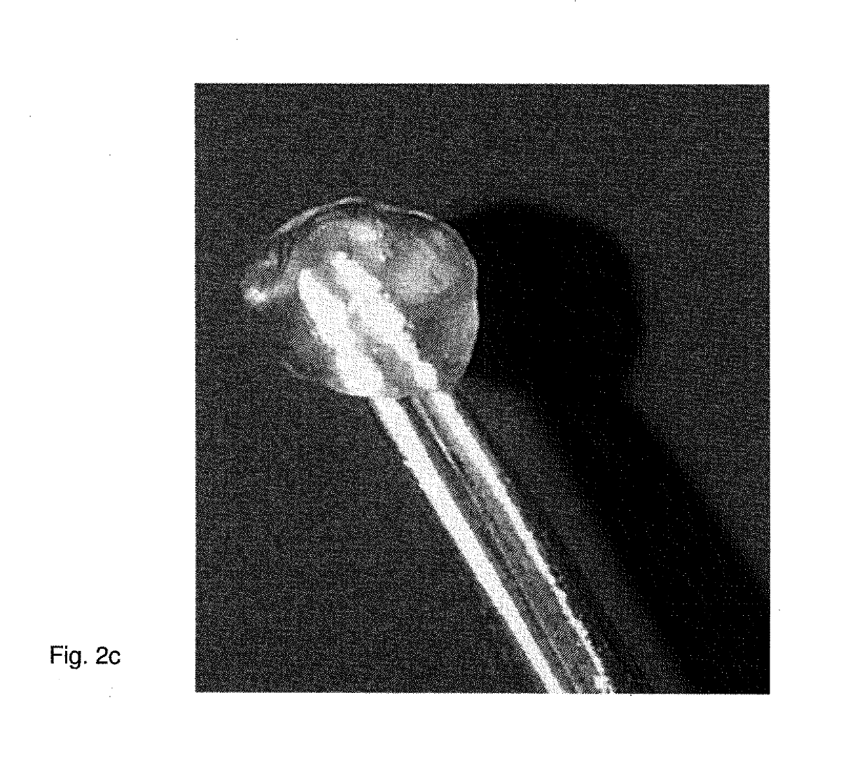

FIG. 2a a schematic illustration of a two-dimensional hydrogel matrix as a series of squares linked together,

FIG. 2b a photographic image of a linked network,

FIG. 2c a photographic image of a linked network,

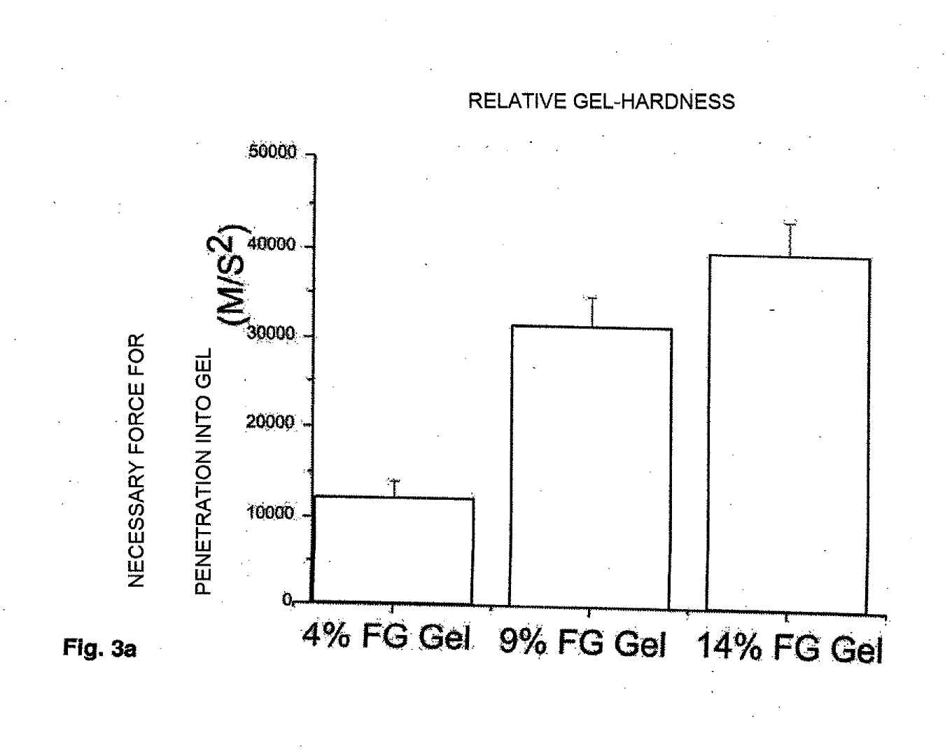

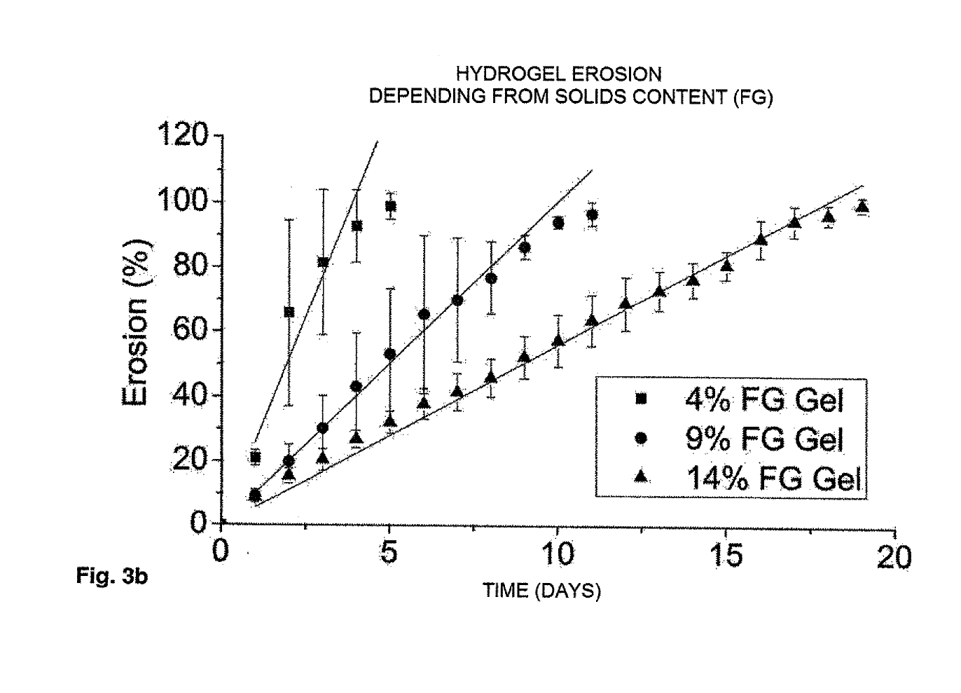

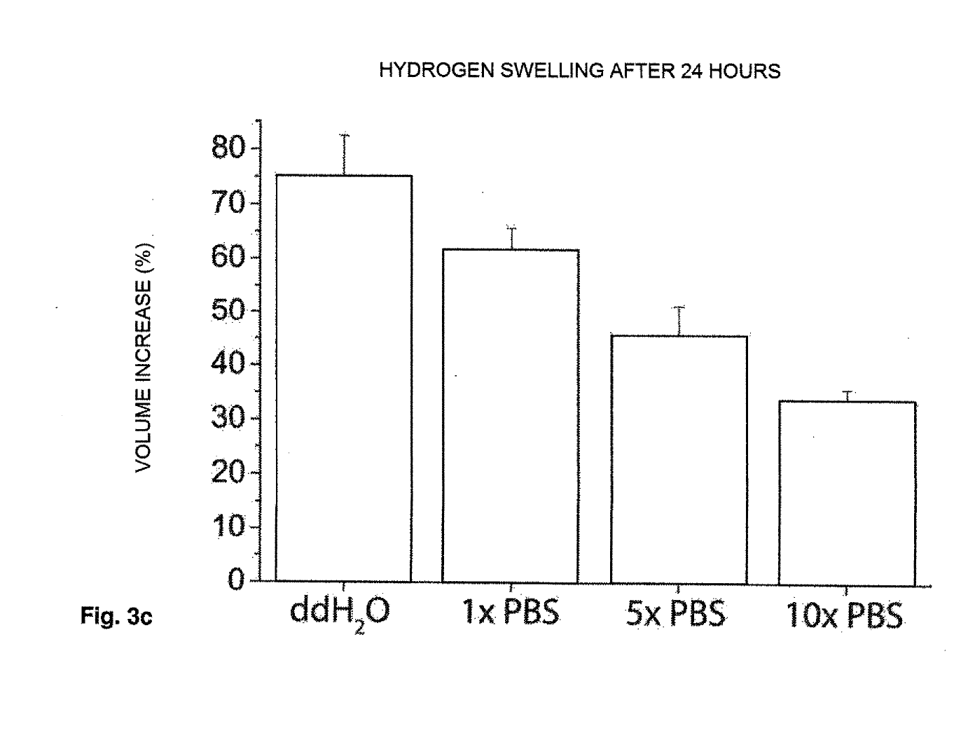

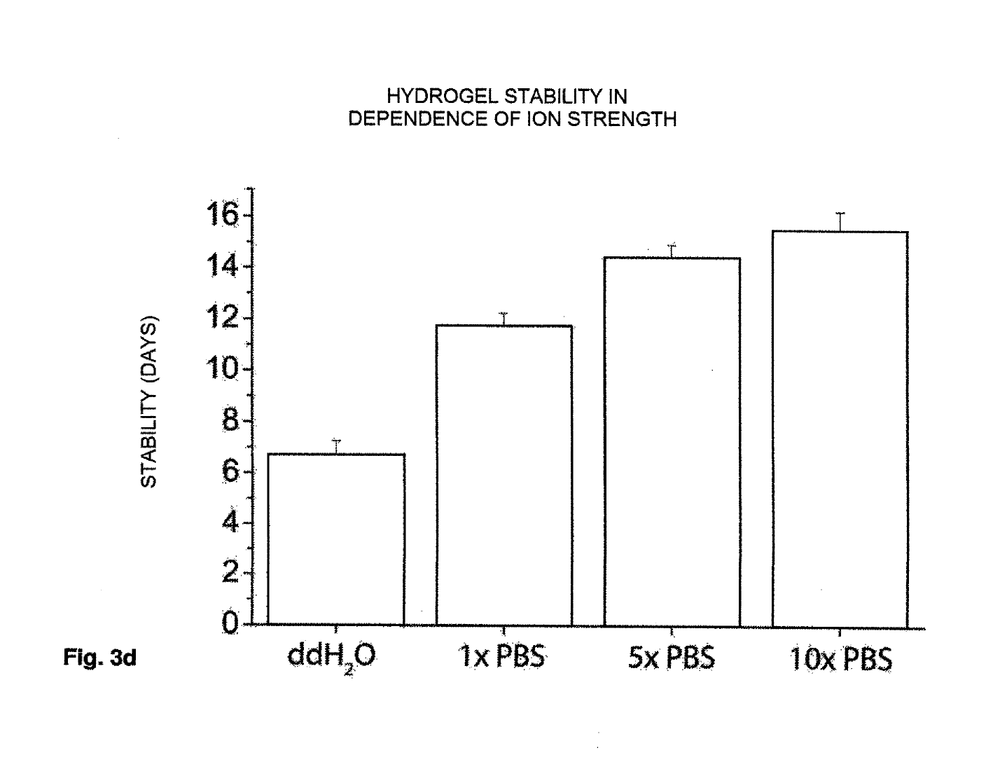

FIG. 3a a diagram showing a comparison of the relative gel hardness of hydrogels having different solids content,

FIG. 3b a diagram showing the hydrogel degradation in dependence on the solids content,

FIG. 3c a diagram showing the hydrogel swelling in dependence of ion strength,

FIG. 3d a diagram showing the hydrogel stability in dependence of ion strength,

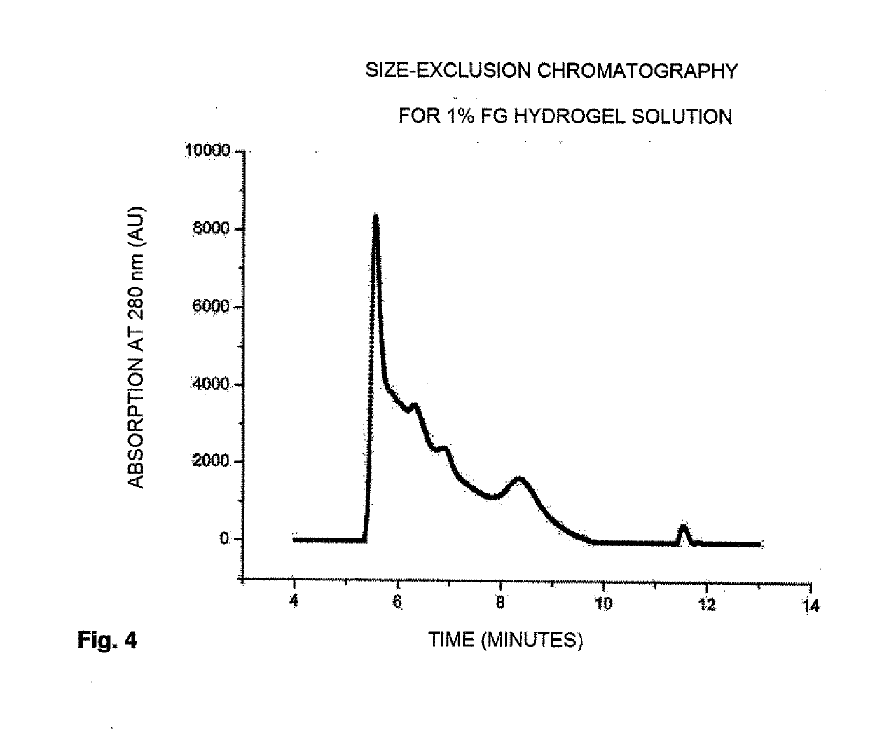

FIG. 4 a size-exclusion chromatography of a hydrogel solution having 1% solids content,

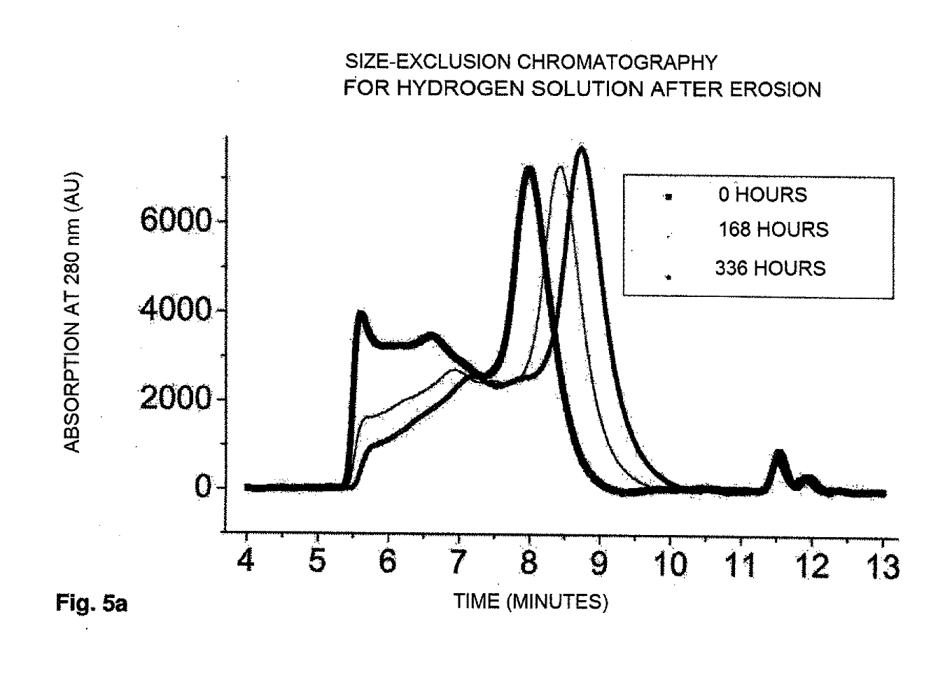

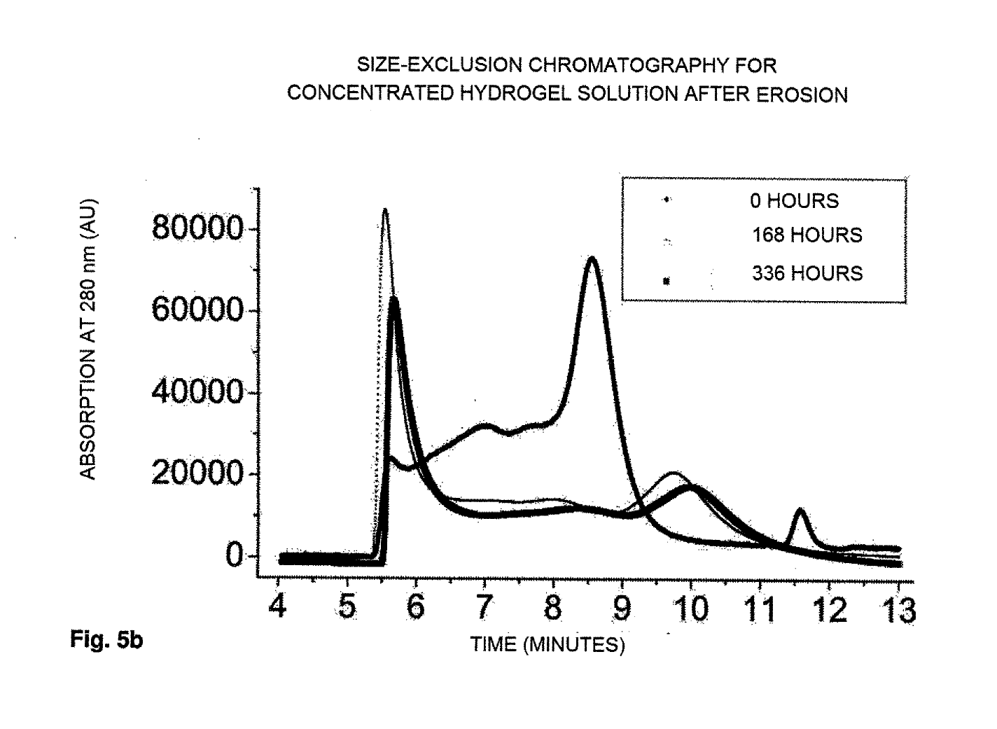

FIG. 5a a size-exclusion chromatography for hydrogel solutions after degradation and reorganizing of particles,

FIG. 5b a size-exclusion chromatography for a degraded hydrogel having 9% solids content after concentrating the probe,

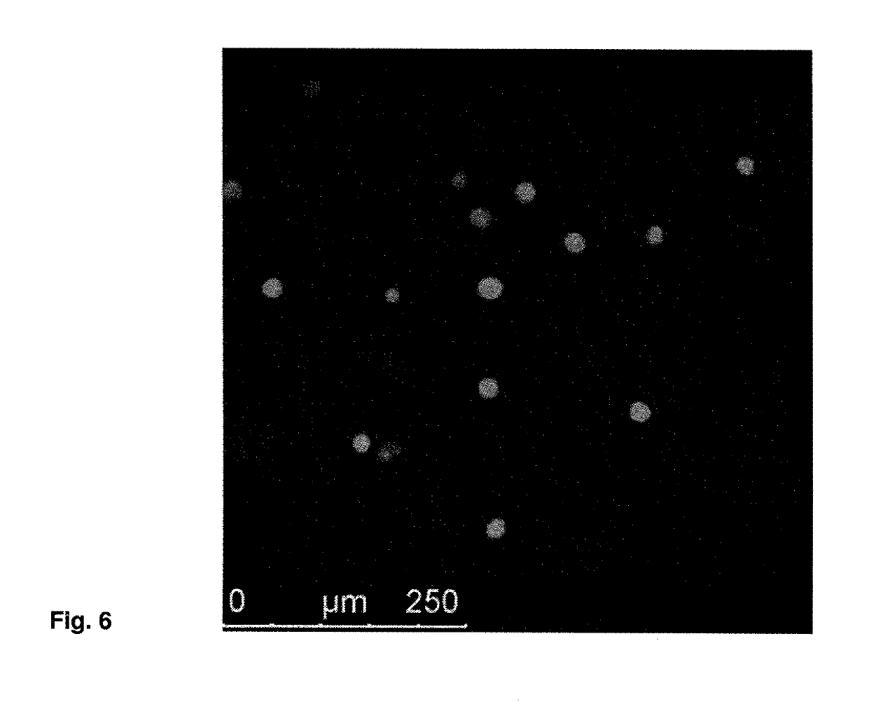

FIG. 6 a photographic image of a live-dead assay,

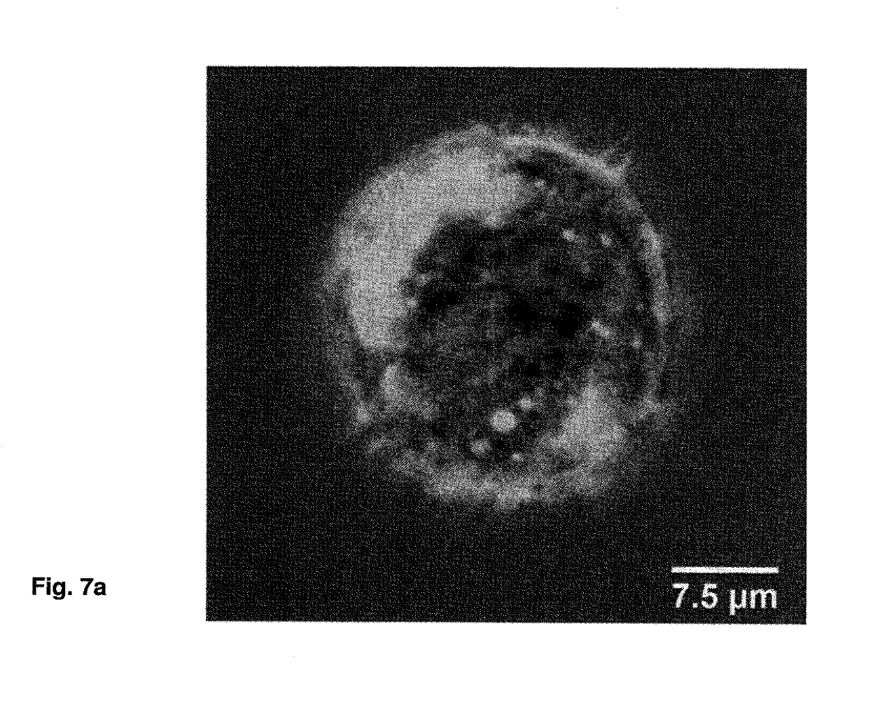

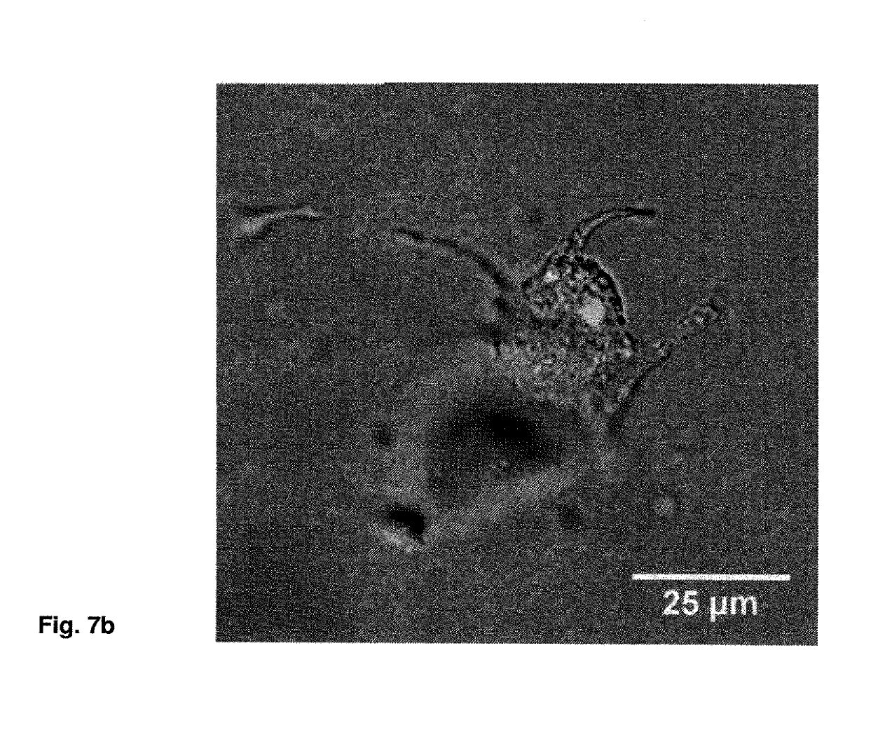

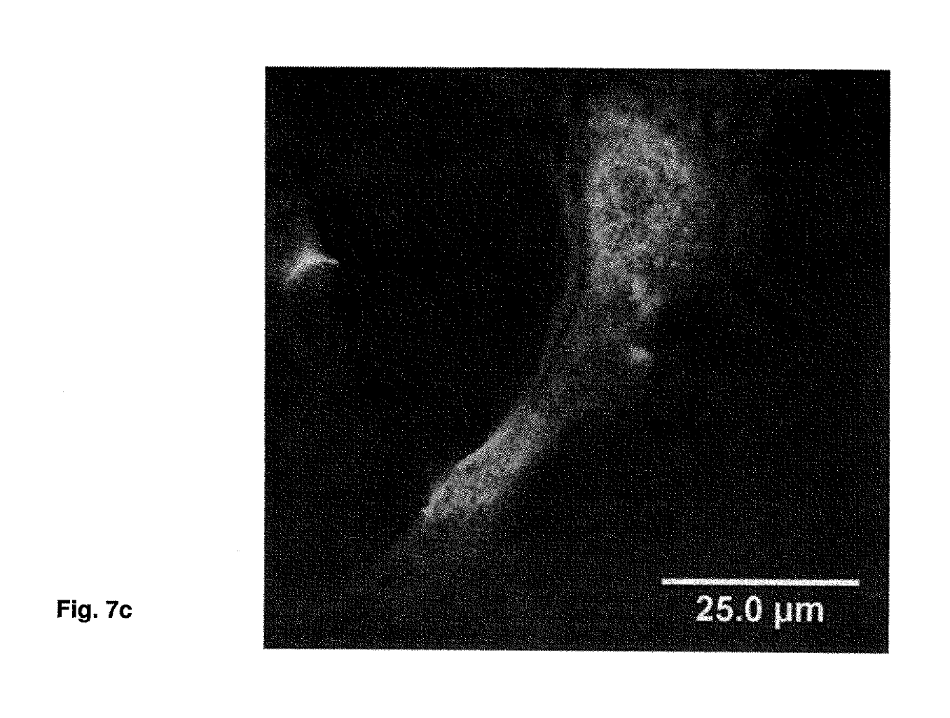

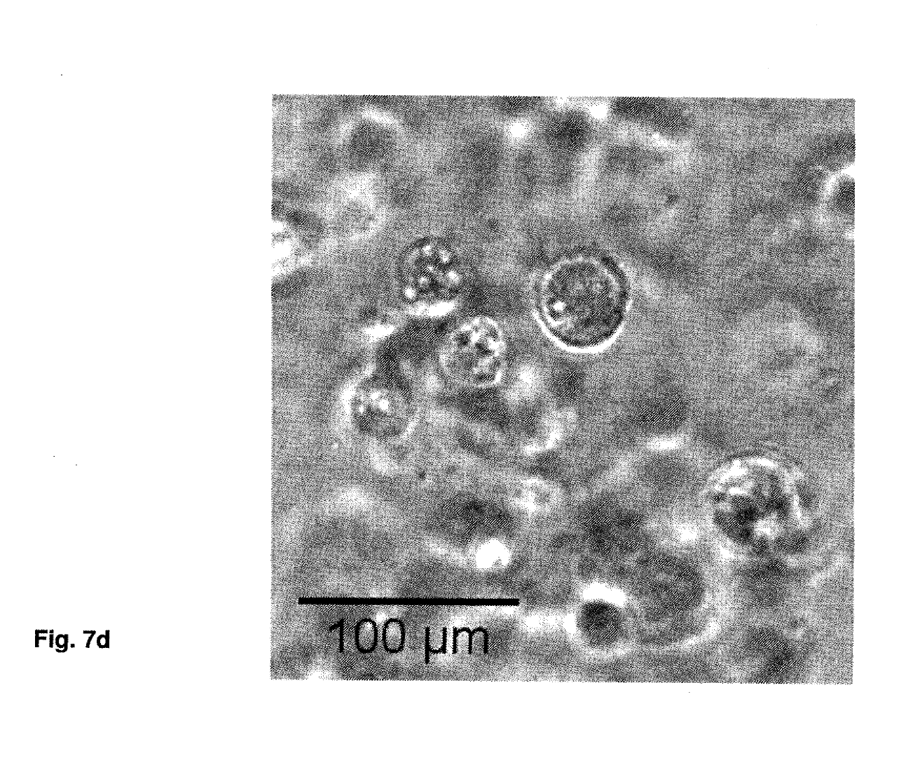

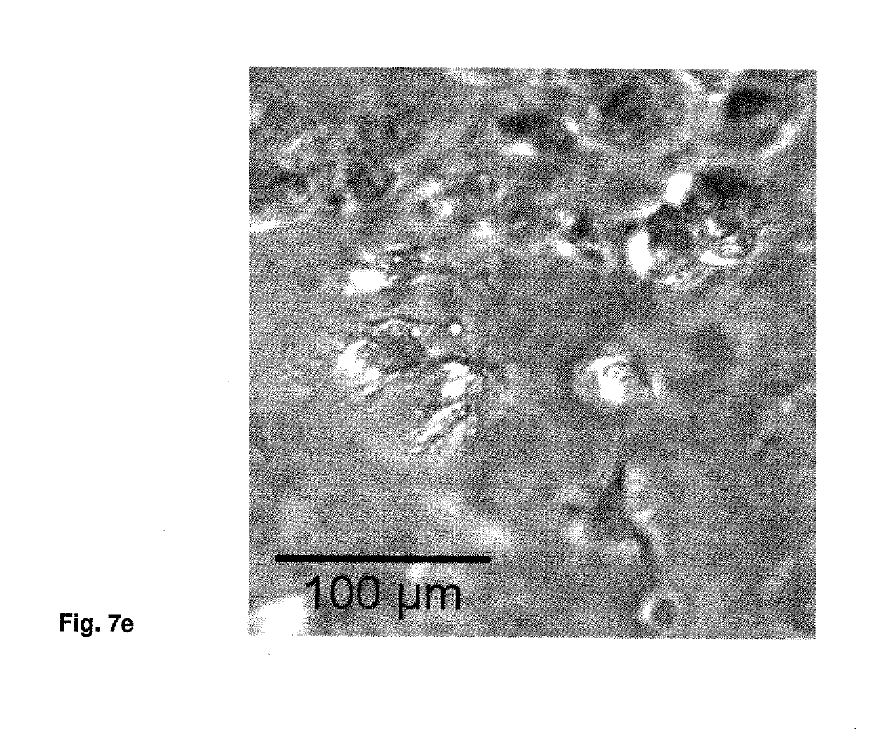

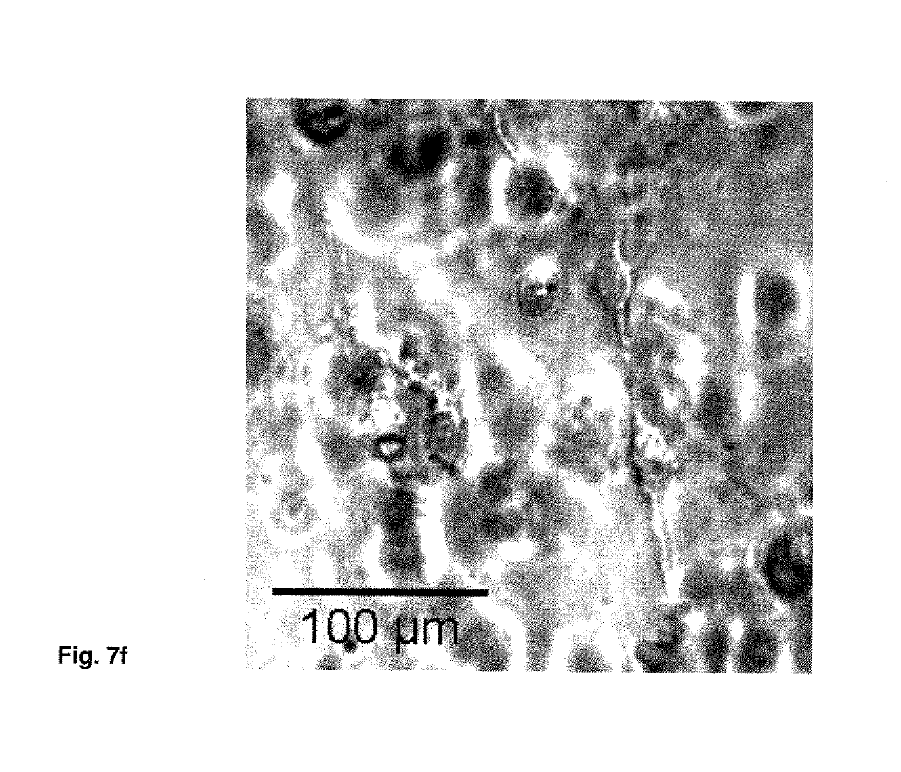

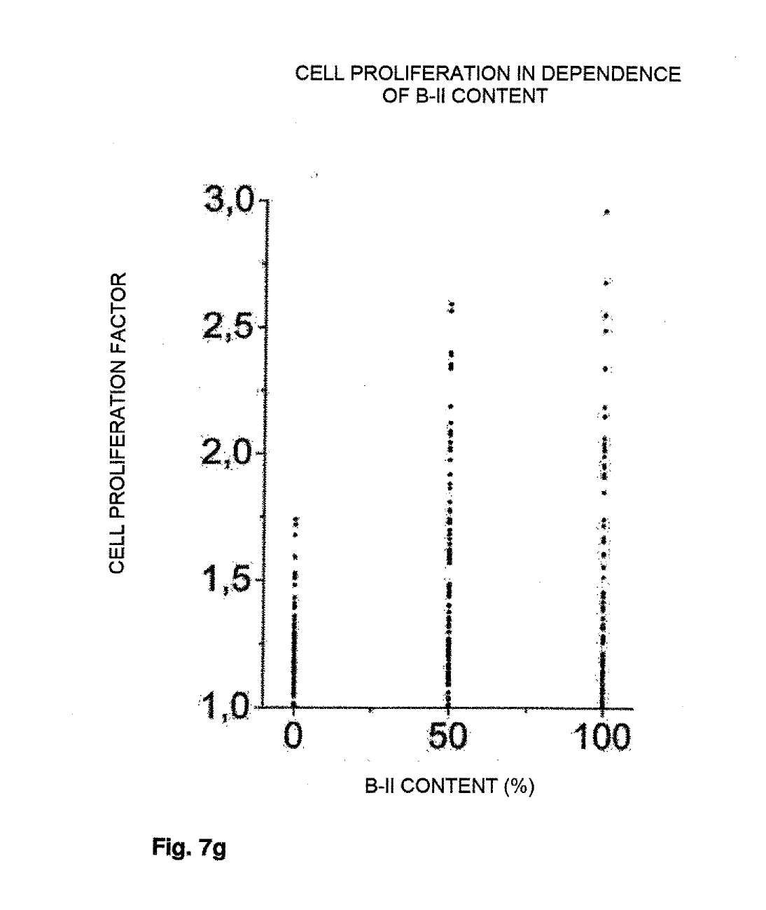

FIG. 7a a microscopic image of a typical cell not propagating itself,

FIG. 7b a microscopic image of cell partially propagating,

FIG. 7c a microscopic image of a cell widely propagating,

FIG. 7d a bright-field microscopic image of the 0% B-II-hydrogel,

FIG. 7e a bright-field microscopic image of the 50%-B-II hydrogel,

FIG. 7f a bright-field microscope image of the 100% B-II-hydrogel,

FIG. 7g a microscopic image of a complete distribution of the cell spreading,

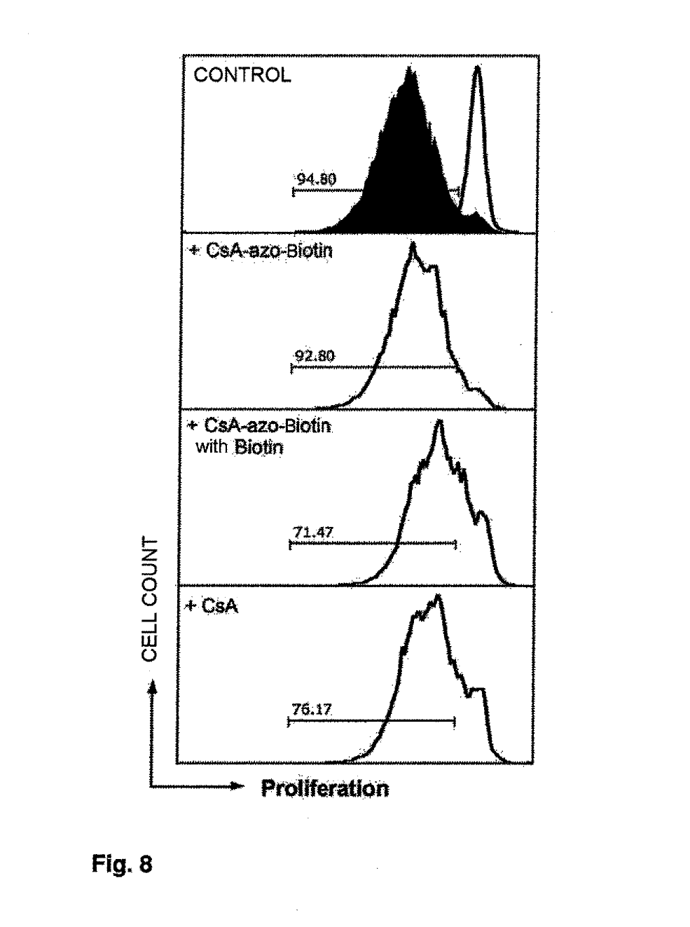

FIG. 8 a transliteration-assay of stimulated mouse T-cells while using flow-through cytometry in dependence of CsA-azo-biotin.

FIG. 1 shows in a schematic illustration a kinetic-controlled hydrogelation based on the protein-ligand interaction. Kinetic control means depending on the volume, only intra-molecular connections of PEG biotin are formed into a protein, for example into avidin, streptavidin or a mutant of these proteins, at low concentrations, see Fig a), small particles with intra- and inter-molecular compounds at higher concentration, see Fig. b), or hydrogels with almost exclusively inter-molecular compounds at highest concentration, see Fig. c), kinetic and thermodynamic control means: Since the compounds are in dynamic rearrangement, that is, kinetics, the stages as shown in Figs. a), b), e) can be transformed each into the other through changes in volume/concentration. The system, (in each stage) also reaches the energetic and entropic minimum, which ends in particle formation or hydrogel formation depending on concentration/volume.

### EXAMPLE I

### Streptavidin/Avidin-PEG-Biotin-Hydrogel

Based on the increased application of non-covalent hydrogel systems for biotechnological applications, a self-organizing hydrogel, which is solely crosslinked through receptor-ligand-interaction between biotin and Avidin, was synthesized.

This model system was utilized to determine the behavior of receptor-ligands based on supra-molecular networks and optimizing them. The hydrogel formation and the erosion are determined by the physical chemistry of receptor-ligand-interaction. The gel formation on the one hand is kinetically controlled and emerges only above the critical concentration c*, on the other hand, based on the combined effects of the ligand-rearrangement, network swelling and entropy degradation occurs faster than expected. A degradation appears as a progressing rearrangement of the gel into ever smaller particles. However, this process is concentration dependent and is reversible through concentration of the degraded hydrogel solution. Finally, for detecting the utilitarian aspect of such a system, peptide motifs were incorporated with the intention to promote stem cell-adhesion and dispersion.

Many hydrogel systems were developed to date on the basis of non-covalent interaction, as for example hydrophobic interaction, protein-protein interaction or peptide-oligosaccharide-interaction. In contrast, a much less utilized crosslinking path is the utilization of simple receptor-ligand-bonds. As network formations are complex processes, until now it was not well understood how the bonding kinetic and bonding energy influence the network formation as well as the resulting physical, mechanical and biochemical properties. The fast binding and the special characteristics, for example the biotin-avidin-bond have been exploited for nano particle formation as well as hydrogel modification- and functionalization; interestingly however, use as a sole crosslinker in a macroscopic network remains largely unexplored. Surprisingly, it was found that this seemingly simple self-organizing net could be only formed under fine-tuned conditions. Although the biotin-avidin-bond is generally viewed as almost irreversible, the physical chemistry of the protein-ligand bond and the dissociation leads to a dynamic network. In addition, the network is easily functionalized, in this case, to influence the morphology of encapsulated mesenchymal stern cells in the 3-D matrix.

The hydrogel-network formation is concentration dependent. In an ideal self-organized grid, avidin tetramers serving as bonding center are crosslinked at both ends by biotinylated linear polyethylene glycol-(PEG)-chains. The structural simplicity of the hydrogel makes it possible to model the matrix two-dimensionally as a series of quadrants that are connected with each other, as shown in FIG. 2a. Due to the steep biotin-avidin-constant Kₒₙ the component concentration becomes decidedly important for the network formation. For the formation of an entire network, the spaces between the avidin binding sites must be similar or smaller than the theoretical average distance of the biotinylated PEG linker ends relative to each other. Upon a suitable topology, a PEG chain can bind two different avidin-tetramers which is fulfilled in the so-called “network-conformation”. The more the concentrations are reduced and the distances increase, the more it is probable that a PEG chain binds itself two times to the same avidin-tetramer in a so-called “closed conformation”, so that the first binding event limits the area while the PEG chain can search for another binding site. Thus, a fragmented, incomplete net is formed which consists of a mixture of network constituents and closed conformation particles, if the intermediate spaces are too large, if only by a bit, to bridge the distances. If the spaces for the PEG are much too large, in order to bridge the distances, a cohesive net will not be formed, but only a distribution of micro- and nano-gel particles with closed conformation edges. Assuming that the PEG 10 kDa-chain is stretched in its contour length of 33.2 nm, a hydrogel with a solids content of 0.21% (27 μM avidin and 54 μM biotinyl-PEG) is formed. However, under these conditions a hydrogel is not being formed, since from the energy point of view the free polymers prefer a semi-helical coil-structure. The median distance of the ends of a 10 kDa-PEG chain was computed through modeling of the polymer using the “model of a wormlike chain”. The calculations show a median end-end distance of 5.0 nm for a 10 kDA-PEG-chain. Assuming an ideal distribution of avidin in solution calculated so that the hydrogel solids content of 9%, which corresponds to 1.27 mM avidin, results in gaps of 5.1 nm between the avidin-tetramers and thus corresponds roughly to the minimum content required to form a cohesive network, as shown in FIGS. 2b and 2c. Although hydrogels can form with a solids content which is as low as 4%, they are less stable as gels with higher solids content due to the patchwork character of the incomplete network, they are markedly softer, as shown in FIG. 3a. Mixtures with a solids content of under 4% form no cohesive network. Thus, the size exclusion-chromatography in FIG. 4 for a gel-solution with 1% solids shows a distribution of small gel particles.

The hydrogel-erosion is a linear and concentration dependent process. Since the biotin-avidin-interaction is generally viewed similarly to a covalent bond as to stability, it was surprising that the erosion of hydrogels in the supernatant occurred first in a time frame of days to weeks, while the hydrogels in the absence of supernatant remains stable over months, as shown in FIG. 3b. By adjusting the solids content, hydrogel stability could be extended from 4 days at 4% solids content to a shelf life of 20 days at 14% solids content. In all cases, hydrogels degrade linearly, typical for physical hydrogels. As is shown in the previous illustration of various possible conformations of the biotinyl-PEG/avidin-interaction shows, it is also possible to describe the physical mechanism for the erosion. When a biotin-avidin-bond breaks out from the network conformation, the PEG-biotinyl-conjugate can bind either an avidin in a network conformation or another available site of the same tetramer by placing itself into a closed conformation which leads to erosion through diffusion from the gel body. Assuming that the bonding energy between avidin and biotin is the same in each network-conformation, the difference between the entropy of the degraded condition and the grid structure is increased in the same manner as the volume of the entire system is increased by adding of buffer solution on the hydrogel. Thus, on the one hand the erosion of the ordered structure in the supernatant is thermodynamically favored; on the other hand, the kinetics of the structural rearrangement is also influenced by the hydrogel swelling as well as the dissociation of biotin from avidin.

Furthermore, the swelling impacts the life span of the hydrogel. The biotinyl-PEG/avidin-hydrogel undergoes a swelling if placed in the supernatant. The swelling is driven by the osmotic pressure—due to the greater concentration of the dissolved material in the interior of the hydrogel as compared to the exterior and ends if either the osmotic equilibrium is reached, or the elastic forces from the expansion of the hydrogel component can resist the osmotic pressure. In order to investigate the effect of the hydrogel swelling, the ion strength of the hydrogel and the supernatant were adjusted. Thus, the hydrogel swelling in de-ionized water (ddH2O) was compared to the hydrogel swelling in various phosphate-buffered sodium chloride solutions (PBS) each in different concentrations, that is, 1×PBS, 5×PBS and 10×PBS. A shown in FIG. 3c by transferring hydrogel from de-ionized water (Milli-Q®-water, ddH2O) in 10× phosphate-buffered sodium chloride solution (PBS), it is possible to reduce the amount of swelling occurring within a time span of 24 hours of a hydrogel having 9% solids content from ˜74% to 35%. Interestingly, the hydrogels with reduced swelling has a longer life span as compared to examples with increased swelling as shown by FIG. 3d. Obviously, the hydrogel swelling contributes to the erosion in two aspects. First, the PEG chains are stretched past their equilibrium length. When a biotin-avidin bond is broken, the PEG will retract into its equilibrium length and is thus more suitable to form bonds in a closed conformation rather than newly forming a network.

Furthermore, the swelling puts the chains under tension through expansion of the polymer past its equilibrium length. The hydrogel with 9% solids content swells in 1 x PBS within 24 hours 60 vol.-% whereby the PEG expands from 5.0 nm to 5.8 nm. Considering the polymer as a wormlike chain, the effective tension was calculated as 3.2 pN due to the expansion of the polymer. The strain speed was about 3.2 pN per hour. Regardless of the biotin-avidin-break resistance, which is oftentimes estimated to be in the area of hundreds of pN, the cited values of high break resistance are realized. According to computations, if a very small strain speed is present, the biotin-avidin bond could be broken by only ˜3 pN of direct force, while measurements with an optical capture at a strain speed of 7.7 pN per second could signify a break at such low force as 3.4 pN. While a firm bond at high strain speed is of highest significance in the field of biotechnology, interestingly, in the hydrogel degradation experiment, the weak interaction of the binding pair is illustrated when stretched at low strain speed. The biotin-avidin-hydrogel is stable under mechanical strain such as ultra sound, while the physical strain resulting from the slow swelling process can disturb the interaction between the receptor-ligand-pair which otherwise is very strong.

The erosion is continuous and reversible. While the hydrogel-erosion is a hydro-dynamically favored process of the closed conformation-particles that diffuse from the hydrogel into the supernatant, it was found that for the eroded hydrogel solutions by means of the size exclusion chromatography, it could be determined that when a cohesive hydrogel vanished, the particles in solution, in time newly reorganize into smaller fragments as also shown in FIG. 5a. It is of great importance that the gelation-/erosion processes are reversible. The eroded hydrogel solutions were re-concentrated to their original volumes, whereby it was found that hydrogels were newly formed within a time frame of 72 hours. This is a relatively long time frame as compared to the rather instant formation of the original hydrogels. In further investigation of this process, 1 ml solution of 30 μl eroded hydrogel with 9% solids content was concentrated to 100 μl, leading to a solids content somewhat below the critical solids content for the hydrogel, and a size exclusion-chromatography carried out. The result of that is shown in FIG. 5b. Directly prior to concentrating the probe the main peak was eluated at 8±0.02 minutes. After 168 and 336 hours after concentrating the probe, the main peak were eluated with the injection peaks. These data show that the network formation is favored at high concentration.

Hydrogels are biocompatible and suitable for 3D-stem cell culture. A fast self-organizing hydrogel is the means for choice for screening of optimal conditions for 3D-cellculture-applications. It was investigated if the cells would be able to survive in the 3D-matrices and to propagate. Modified PEGs were prepared that contained the biotin-peptide-conjugates. The first conjugate biotin-GRGDSPGWC hereinafter called BI contained only the cell adhesion-motif RGDSP. The second conjugate biotin-GRGDSPQGIWGQC, hereinafter called B-II, contained the cell adhesion-motif and also the cleavage point of matrix-metalloprotinase (MMP), PQG↑ ↓IWGQ, so that the cells cleaved the network chemically. The hydrogel-component-solutions were mixed with human mesenchymal stem cells (MSCs) leading to encapsulated cells in a self-organized network. After 24 hours of incubation the hydrogel were tested with a live/dead assay, which showed 98.10% surviving cells (n=157) (See FIG. 6).

It was next investigated whether the hydrogel can realize cell proliferation and growth. First BI and B-II were mixed, that is, a pre-mix was prepared in order to produce hydrogels with 0%, 50% and 100% B-II. A typical immune non-propagating cell is shown in FIG. 7a. A partially propagating cell is shown in FIG. 7b. A widely propagating cell is shown FIG. 7c. Representative bright-field microscopic images of the 0%-, 50%-, 100%-B-II hydrogel are shown in each of FIGS. 7d, 7e and 7f. A complete distribution of the cell proliferation is shown in FIG. 7g. After 48 hours in the 0% hydrogel, a portion of 6.55% of cells show propagating an (n=121), while in the 5% hydrogel a portion of 31.95% of cells (n=131) and in the 100% hydrogel a portion of 25.15% of cells register a proliferation (n=130). The biatinyl-PEG/avidin-hydrogel system is biocompatible and suitable for the cell culture applications with the MMP-cleavable sequence in B-II, which is required for the MSC proliferation.

Presently, a new non-covalent hydrogel is proposed which is cross-linked by the receptor-ligand-interaction between biotin and avidin. The most distinguished feature of this hydrogel is that the hydrogel formation and erosion is determined by the physical chemistry of the receptor-ligand-interaction. On the one hand, the hydrogel formation is a kinetically controlled process and appears only above a critical concentration, on the other hand, as a result of the stretching tension of the polymer chains as well as an increased probability that after breaking the bond during proliferation of the hydrogel, a PEG would take on a closed bond conformation, leads to an erosion of the hydrogel. Likewise interesting was the observation that an erosion, via a progressing rearrangement of the hydrogel particles, results in an increased number of particles having smaller core areas. In addition, this rearrangement is bidirectional and can be reversed via the concentration of the eroded hydrogel. The hydrogel system shows that networks based on receptor-ligand-interaction provides suitable ways for the development of new biomaterials which possess interesting properties. The hydrogels organize themselves under mild conditions and exhibit linear, adjustable erosion rates. The conditions to promote a 3D stem cell culture could be coordinated by researching the synergy effects of the various peptide sequences. As biotinylation is the most widespread method to immobilize biomolecules, the novel system represents a simple way to design highly functionalized bio-matrices and monitor them.

Introduction of the new methods are as follows:

### Preparation of the Hydrogel Component

Avidin (Affiland, Belgium) was obtained as lyophilisized powder. Biotinylated PEG was prepared through dissolving of biotin-NHS- and amino-terminated 10 kDa-PEG (Rapp Polymers, Germany) in a mol ratio of 12:1 in Milli-Q-water with a pH value adjusted to 9.5. The total concentration was 31 mg/mL. The solution was stirred overnight. The reaction mixture was dialyzed in water for two days (8 kDa MW cutoff) to remove the non-linked biotin. Then, the solution was lyophilized.

### Hydrogel Formation

To form the hydrogels, biotinyl-PEG and Avidin in solutions of even volume were dissolved in a molar ratio of 2:1 in the adjusted concentration for the desired solids content. 15 μL were pipetted into a micro centrifuge tube and then placed into a high speed rotary mixer. While mixing, 15 μL biotinyl-PEG-solution were added which led to an almost instant hydrogel formation.

### Hydrogel Stiffness

A table centrifuge was used (5424R, Eppendorf, Hamburg, Germany), to compare small amounts of hydrogel in a high-throughput method. 30 μL hydrogels in 1×PBS were produced. The relative stiffness of the hydrogels were analyzed by means of 45°-centrifuge rotor and penetration of 275 μm-metal balls in dependence of the force issued from the centrifuge.

### Hydrogel-Erosion

In investigating the hydrogel-erosion, the gels were formed as afore-described, with the addition of 10 μL (0.18 nmol) cyanine-5-labeled streptavidin (Invitrogen). Cyanine 5, hereinafter called, Cy5 is a blue fluorescence dye. After the formation of hydrogel 1 ml supernatant was carefully added to the surface of each hydrogel. The gels were then placed into a rotation mixer at 5 rpm per minute and protected from light. Every 24 hours, 125 μl supernatant were taken from each hydrogel and fluorescent-activity measured with a synergy H1-plate reader at 645 nm emission and 675 nm excitation. The supernatant taken was then replaced with 125 μl fresh supernatant and the fluorescence-dates were compared with a standard curve in order to determine erosion.

### Live/Dead Assay

the viability of cells in the PEG-hydrogels were investigated by means of a live/dead assay. Gels that contain cells were rinsed once with PBS. A solution of 10 μM propidium iodide PI) (molecular probes, Invitrogen, Germany) and 0.15 μM fluorescein-diacetate (FDA) (Fluka, Germany) in PBS was applied 3 minutes onto the gels followed by a step of washing with PBS. Images of cells were taken with a confocal microscope (Leica SP5, 10x/0.4). Images were taken at every 10 μm for gel section of 100 μm thickness and the projection of the maximal intensity of the images presented. Brightness and contrast were adjusted in Fidji-Build of ImageJ.

### Cell Proliferation Measurement

48 hours after gel-formation images of various z-levels of the hydrogels were taken. Single cells were identified and provided with ellipses by utilizing Fiji-Build of ImageJ. The eccentricities of the measured ellipses were used to valuate the proliferation. MSCs with eccentricities >1.5 are classified as proliferation.

### EXAMPLE II

### Traptavidin-PEG-Biotin-Hydrogel

Since the erosion of the avidin/streptavidin-hydrogel is triggered by the disassociation of biotin from tetrameric proteins followed by the network-rearrangement, whereby the dissociation speed is reduced, matrix-stability is expected to be improved. While the avidin/streptavidin-biotin-interaction has one of the highest affinities between the protein and a ligand in nature, it was recently reported that the streptavidin-mutant traptavidin produces a 10 times slower dissociation.

In this example, the advantageous biophysical properties of traptavidin are utilized to improve the hydrogel stability. The protein was expressed in E. coli and the purified protein used for the formation of hydrogel with PEG-biotin. As expected, the self-organized hydrogel could be formed only above the critical concentration of 4% solids content. Additionally, the hydrogels remained stable across a time frame of one month without the addition of supernatant.

When the traptavidin-PEG-biotin-hydrogels were incubated with the supernatant, no visible erosion occurred within the time frame of two months. 1% Cy5-labeled streptavidin was added to non-labeled traptavidin to form the hydrogel. In the avidin-hydrogel-system the release of Cy5-labeled streptavidin correlated well with the erosion process and loss of the hydrogel mass while Cy5-labeled traptavidin could be detected during a time frame of 2 months. The result of this experiment demonstrated that the slower dissociation rate between traptavidin and biotin effected a surprisingly drastic increase in stability of the self-organized matrix-network.

### EXAMPLE III

### Streptavidin/Avidin/Traptavidin-DNA-Biotin-Hydrogel

The hydrogel can also be produced by replacing the biotinylated PEG-linker with biotinylated double stranded DNA. Due to the increased stiffness, the DNA-strands reduce the hydrogel swelling and save the ligand at the polymer chain from binding more than once with the same tetrameric protein, which surprisingly in its extent, leads to a substantial reduction of erosion and to an increased shelf life of the hydrogel. The large persistence length of the double-stranded DNA leads to a longer median chain length from one end to the other with an increased contour length as compared to PEG leading to the possibility of producing hydrogels with a reduced amount of avidin. A further advantage is control of the gelation time via heat. The stock solutions can be heated via the melting temperature of the double-stranded DNA and slowly cooled to slow down gel formation.

The DNA-hydrogels were produced by synthesizing in a first step—complementary DNA-single-strands. Each strand is then biotinylated at the 5′ end. The avidin-protein in the gel is being divided into two equal populations and incubated for a short time with one DNA-strand. The solutions are then heated above the melting temperature of the DNA, mixed and then cooled to produce the hydrogel. The gel, as afore-described can be produced as a hydrogel mass or as a thin film. The thin film is produced in that first a small amount of a preheated avidin-DNA solution is pipetted onto the heated surface. An equal amount of the second preheated avidin-DNA solution with the complementary DNA-sequence is being pipetted onto the surface, mixed and distributed across the surface with the pipette to realize a homogenous solution. It is possible to produce films with volumes of solutions below a micro liter, so that mass production of films is cost-effective. The films are also elastic and survive multiple cycles of dehydration and rehydration.

Like the avidin/streptavidin/traptavidin-biotin-PEG-hydrogels, the gel can integrate any biotinylated unit. With this technology, the production of DNA-avidin-hydrogel-arrays can be realized that contain any combination of peptides or other molecules in a broad palette in concentrations as desired by the end user. The hydrogel-matrix is also individually adjustable such that matrices of different concentrations and stiffness can be selected, Possible applications for such a technology include general affinity screening, drug-target screening, optimization assays for the cell culture conditions and the controlled release of the attached charge.

### EXAMPLE IV

### Release of Biomolecules from Hydrogel

Biotinylation represents the most widely used in affinity detection and immobilization of interesting biomolecules. Recently, a covalent hydrogel system was developed in combination with a 2-photon-photochemical reaction which permits the spatial determination of biotinylated substances in 3-D matrices. As compared to the chemical approach of crafting streptavidin to a polymer network, a physical hydrogel which is cross-linked by a streptavidin (and analogs)-biotin-interaction leads to an in situ gelation, ligand mobilization and encapsulation of cells as well as to a broad scope of concentration for a charge with interesting biotinylated substances. Furthermore, the resulting charge density of biotinylated biomolecules can be precisely controlled and determined since it correlates with the ratio between biotinylated polymer and interesting biotinylated biomolecules. Thus, the resulting system leads to an unlimited number of combinations of ligands in a 3D-matrix across a wide concentration area (up to 1 mM) to investigate the cell-ligand interaction in a 3D-environment. Furthermore, the hydrogel can also be used as a release system of biotinylated low-molecular-substances and protein-therapeutics.

The present invention describes a system to solve biotinylated derivatives of the immune-suppressive cyclosporine A (CsA-biotin). The CsA biotin is similarly active as its starting compound CsA, as is also shown in the calcineurin-inhibition-assay as well as in the immune-suppressive assay against Jurkat T-cells, human peripheral mononuclear blood cells and mouse T-cells. When CsA-biotin is embedded in the avidin-biotin-PEG-hydrogel, release of the immune-suppressive effective agent not through the erosion of the hydrogel but controlled through the dissociation of CsA from the avidin-biotin. The hydrogels charged with CsA-azo-biotin were incubated either solely with human serum or incubated at 37° C. with human serum with biotin. After the hydrogels have been finally eroded, the resulting solutions are used to conduct an immune-suppressive assay against mouse-T-cells.

FIG. 8 shows a proliferation-assay with anti-CD3/CD28-antibodies stimulated mouse T-cells while using flow-through-cytometry in dependence of CsA-azo-biotin, released from hydrogel. The control shows the cell proliferation without CsA-effect. The white surface shows stimulated T-cells, the black surface non-stimulated T-cells. The further left the surface is in the diagram, the more proliferation takes place. An amount of 1 μM of the immune suppressive medication CsA inhibited the T-cell proliferation. The T-cells were additionally treated with 1 μM CsA-azo-biotin, that is, biotinylated CsA, which was released from hydrogel. This hydrogel had a solids content of 9% wherein the hydrogel had a molar ratio between avidin and 5 kDa PEG-biotin of 1:2. A difference relative to the control is hardly detectable. With the help of biotin, the bond of CsA-azo-biotin to streptavidin/avidin/mutant can be undone (1 μM CsA-azo-biotin with biotin). This leads to an inhibition of T-cells similar to the positive control CsA. The proliferation was determined after 3 days.

As shown in FIG. 8, the eroded hydrogel in the presence of biotin is as active as the starting compound CsA at the immune-suppression, while the eroded hydrogel in absence of biotin has no immune-suppressive effect. Interestingly, CsA and the biotinylated CsA-analogs have a similar selectivity among the different subtypes of T-cells, while they have no suppressive effect on regulatory T-cells.

Without the addition of biotin the small particles of the eroded hydrogel can engage CsA-biotin and prevent its strong entry into the cells. In comparison, the presence of biotin CsA-biotin can be dissociated from streptavidin and causing an immune-suppressive effect in the cells. Since the CsA-biotin which engages with the eroded hydrogel inhibits the peptidyl-prolyl-cis/trans-isomerase-activity of cyclophilin, the primary target of CsA, the process of the present invention can lead to a controlled function of the effective agent by either inhibiting the biological effect of extra-cellular cyclophilines, or through inhibition of the intra-cellular calcineurins. Moreover, by exchanging biotin with desthiobiotin or iminobiotin, gel-erosion as well as release of active ligands can be accelerated.

1.-12. (canceled)

13. A physical self-organizing hydrogel system for biotechnological applications in the form of a non-covalent network based on protein-ligand interaction, comprising,
a tetrameric protein, biotin or a derivative thereof, a polymer chain of a linear or multi-arm synthetic polymer or a single- or double-strand oligonucleotide, said biotin covalently conjugated to each end of the polymer chain and formed into a biotin-polymer-conjugate cross linked by the tetrameric protein to form the hydrogel network having a solids content relative to the total hydrogel of at least 3%; wherein a mixing ratio between the protein and a number of biotinylated terminal ends of the polymer chain in the biotin-polymer-conjugate is a molar equivalent ratio of 1:2 to 1:8.

14. The physical self-organizing hydrogel system of claim 13, wherein the solids content of the hydrogel is at least 4%.

15. The physical self-organizing hydrogel system of claim 13, wherein the solids content of the hydrogel is at least 6%.

16. The physical self-organizing hydrogel system of claim 13, wherein the solids content of the hydrogel is at least 9%.

17. The physical self-organizing hydrogel system of claim 13, wherein the tetrameric protein for cross linking the conjugates is avidin or streptavidin or a mutant of avidin or of streptavidin.

18. The physical self-organizing hydrogel system of claim 17, wherein the mutant of avidin is traptavidin.

19. The physical self-organizing hydrogel system of claim 13, wherein the biotin derivative is iminobiotin or desthiobiotin.

20. The physical self-organizing hydrogel system of claim 13, wherein the polymer chain is a linear or multi-arm polyethylene glycol (PEG).

21. The physical self-organizing hydrogel system of claim 13, wherein the polymer chain is a branched oligonucleotide with more than two terminal ends.

22. The physical self-organizing hydrogel system of claim 13, wherein the polymer chain is a double-stranded DNA.

23. The physical self-organizing hydrogel system of claim 13, wherein different peptide sequences are inserted into the polymer chain in a modified hydrogel system.

24. The physical self-organizing hydrogel system of claim 13, wherein the polymer-biotin conjugate is selected from the group consisting of biotinylated peptides, biotinylated effective agents, biotinylated oligonucleotides, biotinylated oligosaccharides and biotinylated proteins.

25. A method for producing a hydrogel according to claim 13 comprising the step of mixing the protein and the conjugate to be cross linked in an equivalent ratio of 1:2 to 1:8, wherein the solids content of the hydrogel is at least 3%.

26. A method of using a self-organizing hydrogel system according to claim 13 comprising using the hydrogel system for cultivating cells.

27. A method of using a self-organizing hydrogel system according to claim 13, comprising using the hydrogel system for encapsulating cells.
